(19) United States
(12) Patent Application Publication
(10) Pub. No.: US 2010/0029781 A1
(43) Pub. Date: Feb. 4, 2010

(54) METHODS FOR PREPARATION OF ANTI-ACNE FORMULATION AND COMPOSITIONS PREPARED THEREBY

(76) Inventor: Jerome A. Morris, Erie, CO (US)

Correspondence Address: ALLERGAN, INC., 2525 DUPONT DRIVE, T2-7H, IRVINE, CA, 92612-1599, US

(21) Appl. No.: 12/478,377

(22) Filed: Jun. 4, 2009

Related U.S. Application Data
(60) Provisional application No. 61/058,751, filed on Jun. 4, 2008.

(51) Int. Cl. A61K 31/136; A61P 17/10

(52) U.S. Cl. 514/646

(57) ABSTRACT

The present invention provides methods to make solvent-microparticle (SMP) topical formulations for bioactive drugs. The formulations, which are aqueous gels containing undissolved solid drug, include a drug in a solution which can permeate the stratum corneum layer of the epidermis and the drug in an undissolved microparticulate solid form that does not readily cross the stratum corneum. The solid form is retained in or above the stratum corneum to serve as a reservoir or to provide drug action in the supracorneum zone. The fine, particulate solid component of the invention can confer a smooth, nongritty feel against the skin.

### CROSS-REFERENCE TO RELATED APPLICATION

This application claims the benefit of U.S. provisional patent application Ser. No. 61/058,751, filed on Jun. 4, 2008, and which is incorporated herein by reference in its entirety.

### BACKGROUND

Formulations systems adapted for delivery of bioactive drugs to the skin and via the skin must be designed to address the barrier properties of skin and of skin-related structures, such as lesion surfaces, inflamed skin, scabs, scar tissue, and the like Formulations for drug delivery to or via skin include cosmetic, transdermal, and topical systems. The optimal delivery strategy for administering pharmaceuticals to, and via, the skin varies among various types of formulations depending upon the target tissues. Cosmetic applications, where the target tissue is the skin surface, are designed to provide for negligible drug penetration past the stratum corneum, the layer of dead cells on the surface of the epidermis. For transdermal applications, where the goal is to introduce the drug to the entire body by way of the skin, steady state, high efficiency drug delivery through the epidermal and dermal layers via the capillary bed to the bloodstream and thus systemically to the patient is preferred. However, for topical delivery, minimal systemic absorption is preferred, as the target tissue is at or near the skin surface, and topical agents may furthermore have undesirable side effects when absorbed systemically. However, the bioactive agent must nevertheless penetrate sufficiently to expose the dermal and subdermal tissue to effective doses of the agent, as the target tissue may be several millimeters below the skin surface. For instance, in the treatment of acne, the inflamed sebaceous glands are located in dermal and subdermal layers, but not in deeper musculature. In the treatment of viral lesions such as from Herpes Simplex, viral populations may be similarly located.

In order to adequately dose viable epidermis and dermis, relatively large amounts of drug must cross the intact skin barrier, i.e. the stratum corneum, or the lesional delivery barrier, i.e. scab, plaque, etc., due to the often-broad distribution of the malcondition over a substantial surface area of the body and the need to achieve effective in vivo concentrations of the bioactive drug throughout a tissue layer that can be at least several millimeters in depth. For example, acne, viral skin lesions, fungal infections, and other dermatological disease states can involve substantial areas of the body's surface. Also, it is often advantageous to be able to deliver the bioactive drug over a period of time, such that a desired level of the drug in the target tissue is achieved for a period of time sufficient to achieve the desired result, e.g., killing most of a population of infectious bacterial or fungal cells. Some dermatological conditions, such as acne, require multiple delivery strategies because they have multiple delivery requirements, such as killing skin surface bacteria while also penetrating deep into inflamed sebaceous glands to kill bacteria in that locus.

One topical formulation for the treatment of acne that has found wide acceptance is Aczone®, a topical formulation of the bioactive drug dapsone that is in the physical form of an aqueous gel containing dapsone both in solution and in the solid phase. Commercially available with a 5% concentration of dapsone, this gel material also includes Carbomer 980 as a thickener, methyl paraben as a preservative, diethyleneglycol monoethyl ether (DGME) as a cosolvent, and sodium hydroxide for pH adjustment. A notable feature of Aczone is that the bioactive drug dapsone is not totally dissolved in the vehicle, but also is present in microparticulate, dispersed form. Thus, the Aczone formulation is in the nature of a solvent-microparticle (SMP) topical gel formulation that contains dapsone both in dissolved form and in solid microparticulate form, which is advantageous for treatment of acne, as the dissolved material is readily and immediately available for absorption into dermal and subdermal tissue, while the solid microparticulate form persists on the surface of the skin after application and is only slowly released for absorption. It may be absorbed, for example, by dissolution in skin oils and perspiration and subsequent permeation on a molecular level. The rate of absorption of dapsone from the solid microparticulate state can be controlled, at least in part, by the specifics of the microparticulate form of the solid material, e.g., the size, size distribution, shape, surface/volume ratio, polymorphic crystalline form, and hydration or solvation of the dapsone microparticles. For example, as is well known in the art, larger particles tend to dissolve or disperse more slowly due to the lower surface area/volume ratio in comparison with smaller but similarly shaped particulates.

In U.S. Pat. Nos. 5,863,560 and 6,060,085, topical or dermatological compositions (formulations) containing bioactive drugs such as dapsone or acyclovir, and others, are provided for treatment of various skin diseases. These patents also provide methods of preparation of aqueous gels containing both dissolved and solid particulate forms of the bioactive drug, wherein the drug is at least moderately soluble in at least some organic solvents but only sparingly soluble, or insoluble, in water. These methods involve dissolving the drug in a solvent that has at least some solubility in water, then partially precipitating the drug in solid form by addition of water. This method results in the production of particulates from the sparingly soluble drug whose physical form is governed by the specifics of the technique used to carry out the precipitation, such as concentration, identity of the solvent, relative amount of water added, the presence of other ingredients, the time period over which precipitation occurs, the temperature, post-precipitation handling, and other variables. Many of these variables are likely to be influenced by the scale on which the step of precipitation is carried out, and the degree of control that can be exercised. Therefore, procedures that may work well on a small scale can nevertheless cause problems when attempting to scale up to industrial production of the topical formulation.

### SUMMARY

Embodiment of the invention described herein are directed to novel methods for preparation of solvent-microparticle (SMP) topical formulations including a bioactive drug, and to the formulations prepared by various embodiments the inventive method. A specific example of a drug that is suitable for use in this type of topical SMP formulation is dapsone, which is indicated for treatment of acne, among other malconditions, by topical application. A topical SMP formulation prepared by an embodiment of a method of the invention includes a bioactive agent in two physical states: a dissolved form of a drug that can permeate the stratum corneum layer of the epidermis and become available in tissues of the living dermal layer, and a solid form of a drug that does not readily cross the stratum corneum of the epidermis and thus persists on the exterior surface of the epidermis The solid form can be retained in or above the stratum corneum, where it can serve as a reservoir of a drug for eventual permeation of the skin, or can provide drug action in the supracorneum zone, for example killing bacteria disposed on the skin surface. The solid form can be of a size and form adapted to confer a smooth, soft feeling when applied topically to human skin. The solid form of the drug may be any one of multiple polymorphic forms of a single drug, or can include more than one polymorph.

In various embodiments of the invention, a method of preparing a solvent-microparticle topical gel formulation comprising a bioactive drug, wherein the formulation comprises the drug dissolved in a liquid and the drug in a microparticulate solid form dispersed in the liquid, the method comprising first forming the liquid by combining an organic solvent and water, and then contacting the drug in a microparticulate solid form with the liquid, such that the microparticulate solid form does not entirely dissolve in the liquid; and dissolving a thickener in the liquid at a concentration sufficient to form a gel, is provided.

In another embodiment of the invention, a method of preparing a solvent-microparticle topical gel formulation comprising a bioactive drug is provided wherein, prior to the step of contacting the microparticulate solid form with the liquid, forming a solution of the drug in the liquid, wherein the drug is substantially completely dissolved in the liquid.

In another embodiment, a topical SMP formulation prepared by a method of the invention is provided.

In another embodiment, a second drug can be included in a topical SMP formulation prepared by a method of the invention In various embodiments, methods of preparing a topical SMP formulation of the invention comprising a second drug are provided.

In various embodiments, the amount of the drug in microparticulate solid form dispersed in a unit volume of the liquid is no more than about six times the amount of the drug dissolved in the unit volume of the liquid.

In various embodiments, the topical composition is a semi-solid aqueous gel, wherein a drug is dissolved in the gel such that the drug has the capacity to cross the stratum corneum layer of the epidermis and become available at least in the living dermal tissue, and wherein the composition also contains the drug in a microparticulate state that does not readily cross the stratum corneum of the epidermis In various embodiments, the topical composition is a semi-solid or gel-like vehicle that can include a preservative, active surfactants or emulsifiers antioxidants, or sunscreens, or any combination thereof

In some embodiments, the solid form of the active agent is a amorphous solid. In other embodiments, the solid form of the active agent is a flake. In still other embodiments, the solid form of the active agent is a crystal. In various embodiments, the invention provides compositions with desirable physical properties, such as a smooth, non-gritty feeling against the skin of a patient.

### DETAILED DESCRIPTION

As used herein, “dapsone” refers to the chemical compound dapsone having the elemental formula C₁₂H₁₂N₂O₂S, structure

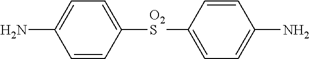

known as bis(4-aminophenyl)sulfone, including its hydrates, solvates, tautomers, and salts; also known as 4,4′-sulfonylbisbenzeneamine, 4,4′-sulfonyldianiline, and diaphenylsulfone; and dapsone analogs; and dapsone related compounds. “Dapsone analogs” refers to chemical compounds that have similar chemical structures and thus similar therapeutic potential to dapsone such as the substituted bis(4-aminophenyl)-sulfones. “Dapsone related compounds” refers to chemical compounds that have similar therapeutic potential, but are not as closely related by chemical structure to dapsone such as the substituted 2,4-diamino-5-benzylpyrimidines.

A “drug,” “active agent,” “bioactive agent,” or “pharmaceutical,” as the terms are used herein, refer to a medicinal compound, organic, organometallic, or inorganic, that can be used for treatment of a malcondition wherein topical application of the material is medically indicated.

As used herein, “gel” refers to a colloid in a more solid form than a solution; a jelly-like material formed by the coagulation or gelation of a colloidal liquid; many gels have a fibrous matrix and fluid filled interstices: gels can be viscoelastic as well as viscous, and in various embodiments gels can resist some mechanical stress without deformation.

As used herein, the term “microparticulate” or “microparticle” refers to any solid form of an active agent, including dapsone, provided that the average particle size is on the micron scale, that is, less than 1 mm, and that there are substantially no particles of size larger than 1 mm in a sample of the solid. By “average particle size” is meant an average of the particle diameters of all the particles in a population of the particles. By “particle diameter” of an individual particle is meant, if the particle is substantially spherical, the diameter of the sphere; if the particle is elongated or of irregular shape, an average of diameters along all axes. The average particle size can be on the order of microns (1-10 microns), tens of microns (11-100 microns), or hundreds of microns (101-999 microns), or it can be submicron. Typically, average particle sizes in an SMP formulation of the invention are around 10-500 microns. The microparticulate active agent described herein can be in any solid shape, such as flakes or crystals or amorphous particles.

By the terms “dissolved” or a “solution” is meant a molecular solution of a substance, the substance being a solid in pure form at room temperature, in a liquid, wherein individual molecules of the substance are separated from each other in the liquid solution, as is well known in the art. Few if any long-lasting interactions between molecules of the substance take place in the solution phase, and the molecules of the substance are surrounded by molecules of the materials making up the liquid.

By the terms “suspended,” “suspension,” “dispersed,” and “dispersion” are meant a physical state wherein finely particulate solid particles are mixed with a liquid, but are not dissolved in the liquid. There are many significant and long-term associations between individual molecules of the suspended or dispersed substance within the particles. Molecules of the substances making up the liquid may permeate the particles, but the particles retain a cohesive structure, wherein aggregations of molecules of the solid substance persist. Upon standing, these particles may be acted on by the force of gravity, causing them to accumulate at the bottom of a vessel containing the suspension or dispersion.

The microparticulate solid can be any polymorph of a given drug, or can be a mixture of multiple polymorphs. It can include hydrates, solvates, tautomers, salts or molecular complexes of the drug. By a “molecular complex” of a drug is meant a form of the drug wherein the active molecule is in a defined molecular association with a carrier, for example a cyclodextrin complex of a drug. For example, when the drug is dapsone, various polymorphic forms such as Form I or Form III can be used.

The microparticulate solid may have been milled or ground to achieve smaller sized particles. As used herein, the terms “milling” and “grinding” refer to the action of breaking a solid material into smaller pieces. The grinding of solid matters occurs under exposure of mechanical forces that trench the structure by overcoming of the interior bonding forces. After the grinding the state of the solid is changed: the grain size, the grain size disposition and the. grain shape.

As used herein, “preservative” refers to any substance which prevents bacterial growth, mold growth, fermentation, oxidation, or molecular decomposition, or any combination thereof.

“Therapeutically effective amount” refers to an amount of a drug, or a combination of more than one drug or an amount of a formulation including the drug or the combination, effective to treat dermatological condition in a patient.

The term “topical” as used herein refers to the route of administration of a dermatological composition that involves direct application to the exterior body part being treated, the skin, or a lesion on the body exterior where skin has decomposed such as a scab, plaque or open sore. Typically, areas of the body suitable for application of the dermatological composition include the skin of the face, throat, neck, scalp, chest, back, ears, and other skin sites. Application to mucosal surfaces is not included in the term “topical” as used herein.

As used herein, the term “treat”, “treatment”, or “treating” includes prophylaxis of the specific disorder or condition, or alleviation of the symptoms associated with a specific disorder or condition and/or preventing, ameliorating, inhibiting or eliminating the symptoms.

Embodiments of the invention described herein provides topical SMP gel formulations and methods to prepare the formulations. Embodiments of the topical SMP formulations include a liquid component, the liquid component including a mixture of water and an at least partially water-soluble solvent. The solvent can be an organic solvent, for example the solvent can include diethyleneglycol monoethyl ether (DGME), N-methylpyrrolidone (NMP), N,N-dimethylformamide, N,N-dimethylacetamide (DMA), dimethylsulfoxide (DMSO), or any other substantially non-toxic solvent suitable for application to human skin, wherein the solvent has at least some water solubility. Or, combinations of any of these solvents can be used. Additional examples include ethanol, propylene glycol, glycerol, diethyleneglycol, triethyleneglycol, polyethylene glycol, propylene carbonate, pyrrolidone, N-methyl pyrrolidone, dimethylsulfoxide, triethanolamine, 1,4-butanediol, triacetin, diacetin, dimethyl isosorbide, and the like, alone or in combination. The solvent and the water can be present in various relative amounts in the liquid. The solvent need not be miscible with water in all proportions, but when mixed at the particular ratio selected for a formulation, the water and the solvent should form a single phase. at room temperature.

Water is typically the predominant component of the liquid. For example, the solvent can make up about 10-40% of the liquid by weight, with the remainder of the liquid component as described herein being water. Deionized water or distilled water can be used in a method of the invention. The water can be sterilized, for example by ultrafiltration or by boiling, to remove any infectious organisms that could be present. The water can be substantially free of dissolved solids, such as salts or other contaminants. USP grade water can be used.

Other solvents that can be used in conjunction with water to form the liquid of the inventive method include, but are not limited to: benzyl alcohol, denatured alcohol, methanol, isopropyl alcohol, water, propanol, acetone, chlorobutanol, methyl ethyl ketone, sorbitan monolaurate, sorbitan monooleate, sorbitan monopalmitate, butanol, butyl alcohol, diglycerides, dipropylene glycol, eugenol, diacetin, diethanolamine, monoacetin, monoglycerides, PEG vegetable oil, N,N-dimethylformamide, N-methyl formamide, N-methylacetamide, N,N-dimethylacetamide, or combinations thereof.

Glycol ethers are organic solvents that are moderately soluble to miscible with water and can be as a solvent in formation of a liquid used in a method of the invention. A glycol ether is an ether formed from at least one glycol and at least one lower alkyl alcohol. Preferably the glycol is selected from an alkylene glycol such as ethylene glycol, propylene glycol, or butylene glycol. The ether portion of the glycol ether is a radical of a lower alkyl alcohol such as a C₁ to C₆ alcohol. Preferably, the ether portion alcohol is selected from methyl alcohol, ethyl alcohol, propyl alcohol, isopropyl alcohol, butyl alcohol, or isobutyl alcohol.

Examples of glycol ethers under the classification of ethylene glycol ethers include ethylene glycol monopropyl ether (propoxyethanol), ethylene glycol monobutyl ether (butoxyethanol), diethylene glycol monoethyl ether (ethoxydiglycol, DGME), diethylene glycol monobutyl ether (butoxydiglycol), diethylene glycol monoisopropyl ether (isopropyldiglycol), and diethylene glycol monoisobutyl ether (isobutyl diglycol).

Glycol ethers under the classification of propylene glycol ethers include propylene glycol monomethyl ether, dipropylene glycol monomethyl ether (PPG-2 methyl ether), tripropylene glycol monomethyl ether (PPG-3 methyl. ether), propylene glycol n-propyl ether, dipropylene glycol n-propyl ether (PPG-2 propyl ether), propylene glycol monobutyl ether, dipropylene glycol monobutyl ether (PPG-2 butyl ether), propylene glycol monoisobutyl ether, and dipropylene glycol dimethyl ether. In one embodiment of the invention the solvent is ethoxydiglycol. In another embodiment, the solvent is methoxydiglycol. Additional suitable exemplary glycol ethers are disclosed, e.g., in Aldrich Handbook of Fine Chemicals, 2003-2004 (Milwaukee, Wis.).

In one embodiment, formulations of the invention can have a glycol ether. present in about 20.0 wt. % to about 40.0 wt. %. In another embodiment, formulations of the invention cab have a glycol ether present in about 20.0 wt. % to about 35.0 wt. %. In another embodiment, formulations of the invention can have a glycol ether present in about 25.0 wt. % to about 40.0 wt. %. In yet another embodiment, formulations of the present invention can have a glycol ether present in about 25.0 wt. % to about 35.0 wt. % of the composition. More specifically, compositions of the present invention have a glycol ether present in about 25.0 wt. % of the composition.

A drug is present in the SMP formulation, both dissolved and dispersed in the liquid component. An example of a drug for use in a method of the invention is dapsone. Another example is acyclovir or ganciclovir. A drug is present in the topical SMP formulation in two distinct physical forms. First a solution or dissolved form of the drug is present in the SMP formulation, wherein the drug substance is dissolved in the liquid comprising the water and. the solvent. Therefore the drug has at least a limited solubility in the liquid of the SMP formulation. This dissolved form of the drug can permeate the stratum corneum layer of the epidermis and become available in the living dermal tissue when the formulation is applied to human skin. The second physical form of the drug in the SMP formulation is a microparticulate solid form that is not dissolved in the liquid, but rather is dispersed or suspended in the liquid of the formulation. Therefore the drug is not completely soluble in the liquid comprising water and a solvent at the concentration of drug and the composition of liquid used. The formulation can be in gel form due to the presence of a thickener as discussed below. This solid microparticulate form does not readily cross the stratum corneum of the epidermis when the formulation is applied to human skin. Instead, the solid form is retained in or above the stratum corneum to serve as a reservoir for eventual absorption through the stratum corneum into the living dermal tissue, or to provide drug action in the supracorneum zone, or both. The fine, microparticulate solid component can confer a smooth, nongritty feel against the skin. For example, flakes or amorphous solids of relatively small average particle diameter can provide a smooth skin feel or texture.

Examples of drugs that can be used in a formulation prepared by a method of the invention include, in addition to dapsone, acyclovir, and ganciclovir: salicylic acid, resorcinol, resorcinol acetate, benzoyl peroxide, sulfur, retinol, retinoic acid, citric acid, an alpha hydroxy acid, retinal, pharmaceutically acceptable salts thereof, and combinations thereof. Specifically, the active agent can be at least one of adapalene, azaleic acid, erythromycin salnacedin, inocoterone acetate, or isotretenoin anisatil.

In various embodiments of the invention, the drug can be a glucocorticoid. Glucocorticoids include, e.g., betamethasone dipropionate, betamethasone valerate, clobetasol propionate, diflorasone diacetate, halobetasol propionate, amcinonide, desoximetasone, fluocinonide, fluocinonide acetonide, halcinonide, triamcinolone acetonide, flurandrenolide, hydrocortisone valerate, hydrocortisone butyrate, mometasone furoate, aclometasone dipropionate, desonide, dexamethasone sodium phosphate, and fluocinolone acetonide.

In various embodiments of the invention, the drug can be calcipotriene, a retinoid, anthralin, coal tar, salicylic acid, or a combination thereof.

In various embodiments of the invention, the drug can be an antibiotic agent. As used herein, an “antibiotic agent” refers to any compound having activity against either Gram-positive or Gram-negative organisms (i.e., inhibits. the growth or destroys the development of either Gram-positive or Gram-negative organisms). Stedman's Medical Dictionary, Illustrated, (25th Ed.), Williams & Wilkins: Baltimore (1990) and Mosby's Medical, Nursing, & Allied Health Dictionary, (5th Ed.), Mosby: St. Louis (1998).

Any suitable antibiotic agent can be employed, provided the antibiotic. agent effectively inhibits the growth or destroys the development of either Gram-positive or Gram-negative organisms and the antibiotic agent remains stable in the formulation. Preferably, the stability is over a prolonged period of time, e.g., up to about 3 years, up to about lyear, or up to about 6 months, typically experienced in the manufacturing, packaging, shipping, and/or storage of the composition. Suitable antibiotic agents are disclosed, e.g., in Physician's Desk. Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989;. University of Wisconsin Antimicrobial Use Guide, http://www.medsch.wisc.edu/clinsci/amcg/amcg.html; Introduction on the Use of the Antibiotics Guideline, Descriptions of Specific Antibiotic Classes, Thomas Jefferson University, http://jeffline.tju.edu/CWIS/OAC/antibiotics_guide/intro.html; and references. cited therein.

Suitable classes of antibiotic agents include, e.g., β-lactams, aminoglycosides, antifungal agents, and combinations thereof. Suitable antibiotic agents include, e.g., cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin. indamyl, carbenicillin, mezlocillin, piperacillin, ticarcillin, cloxacillin, dicloxacillin, floxacillin, methicillin, nafcillin, oxacillin, colistmethate, polyrnixin b, trimethoprim, cotrimoxazole, mafenide, sulfadiazine, sodium sulfacetamide, sulfacytine, sulfadiazine, sulfamethoxazole, sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, ascariasis, pinworm, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, pharmaceutically acceptable salts thereof, and combinations thereof Specifically, the antibiotic agent can be dapsone, erythromycin, tetracycline, clindamycin, cephalosporin, pharmaceutically. acceptable salts thereof, or a combination thereof. In a preferred embodiment, the antibiotic agent is dapsone. When the compositions are in use (i.e., when the. composition is placed upon the skin of a patient (e.g., human)), the dapsone can be in continuous contact with the skin surface of the patient.

Specifically, the antibiotic can be at least one of Arnphomycin, Apramycin, Avilamycin, Azithromycin, Bacitracin, Bactiracin Zinc, Clarithromycin, Clindamycin, Clindamycin Hydrochloride, Clindamycin Palmitate Hydrochloride, Clindamycin Phosphate, Dirithromycin, Erythromycin, Erythromycin Acistrate, Erthromycin Estolate, Erthryomycin Ethlylsuccinate, Erthryomycin Gluceptate, Erythromycin Lactobionate, Erthromycin Propionate, Erthromycin Stearate, Fosfomycin, Fosfomycin Tromethamine, Josamycin, Kitasamycin, Lexithromycin, Lincomycin, Limcomycin Hydrochloride, Metronidazole Hydrochloride, Metronidazole Phosphate, Mirincamycin Hydrochloride, Paldimycin, Paulomycin, Pirlimycin Hydrochloride, Ranimycin, Relomycin, Roxithromycin, Spectinomycin Hydrochloride, Spiramycin, Stallimycin Hydrochloride, Tobramycin, Vancomycin, Vancomycin Hydrochloride, Zorbamycin, Mupirocin, Mupirocin Calcium, and Parachlorophenol.

Specifically, the active agent can also be an acidic compound. An acidic compound is one that contains an organic acid group or is at least weakly acidic in an aqueous-based solution and can be more effective in its protonated form. Examples of acidic compounds include salicylic acid, retinoic acid, and azelaic acid.

The active ingredient can be an antiseptic. As used herein, an. “antiseptic” is an agent or substance capable of effecting antisepsis, i.e., the. prevention of infection by inhibiting the growth of infectious agents. Stedman's Medical Dictionary, 25th Ed., illustrated, Williams & Wilkins, Baltimore, Md., p. 100 (1990). Any suitable antiseptic can be employed, provided the suitable antiseptic effectively inhibits the growth of infectious agents. Suitable antiseptics include, e.g., triclosan, phenoxy isopropanol, chlorhexidine. gluconate, povidone iodine, and any combination thereof. The antiseptic can be. employed in any suitable amount, provided the suitable amount antiseptic effectively inhibits the growth of infectious agents. For example, the antiseptic can be employed up to about 20 wt. % of the of the composition, or up to about 10 wt. % of the of the composition, or up to about 5 wt. % of the composition.

Calcipotriene is a synthetic topical form of vitamin D. It is involved in the growth and development of skin cells. Topical calcipotriene is used to treat plaque psoriasis (psoriasis with scaly patches). Chemically, calcipotriene is (5Z,7E, 22E,24S)-24-cyclopropyl-9,10-secochola-5,7,10(19), 22-tetraene-1 alpha, 3 beta, 24-triol-, with the empirical formula C₂₇H₄₀O₃.

Retinoids include vitamin A or vitamin A-like compounds, including, but. not limited to, retinoic acid (RA), a natural acidic derivative of vitamin A. Retinoids play a critical role in normal development, growth and differentiation by modulating the expression of target genes.

Anthralin is an anthraquinone (the 9, 10 quinone derivative of anthracene; anthraquinones can be made synthetically and also occur in naturally in aloe, cascara sagrada, senna, and rhubarb; the antineoplastic mitoxantrone is a synthetic derivative) derivative that reduces DNA synthesis and mitotic activity in hyperplastic epidermis, restoring the normal rate of epidermal cell proliferation and keratinization; used topically in the treatment of psoriasis and other skin conditions (also called dithranol).

Coal tar is a viscous black liquid containing numerous organic compounds that is obtained by the destructive distillation of coal. Coal tar can be distilled into many fractions to yield a number of useful organic products, including benzene, toluene, xylene, naphthalene, anthracene, and phenanthrene. These substances, called the coal-tar crudes, form the starting point for the synthesis of numerous products-notably dyes, drugs, explosives, flavorings, perfumes, preservatives, synthetic resins, and paints and stains. Coal tar is used medically to treat eczema, psoriasis, seborrheic dermatitis, and other skin disorders.

Salicylic acid is 2-hydroxybenzoic acid (C₆H₄(OH)CO₂H), which is a colorless, crystalline organic carboxylic acid. Salicylic acid is used to treat many skin disorders, such as acne, dandruff, psoriasis, seborrheic dermatitis of the skin and scalp, calluses, corns, common warts, and plantar warts.

The topical compositions for use in the invention described herein include single agents, or agents in combination. In one specific embodiment of the invention, the active agent can be co-administered with photochemotherapy with ultraviolet A (PUVA). In another specific embodiment of the invention, the active agent can be co-administered with phototherapy with UVB. As used herein, “photochemotherapy with ultraviolet A (PUVA)” refers to a type of ultraviolet radiation treatment (phototherapy) used for severe skin diseases. PUVA is a combination treatment which consists of Psoralen (P) administration. and then exposure of the skin to long wave ultraviolet radiation (UVA). Psoralens include compounds which make the skin temporarily sensitive to UVA. As used herein, “phototherapy with UVB” refers to a type of radiation. treatment or therapy involving exposure to ultraviolet B light (wavelength 280-315 nm).

Any suitable amount of active agent can be employed, provided the amount of agent employed effectively treats the dermatological condition and the effective amount of the active agent remains stable in the composition over a prolonged period of time, and provided that the solubility of the drug or active agent. Preferably, the stability is over a prolonged period of time, e.g., up to about 3 years, up to about 1 year, or up to about 6 months, typically experienced in the manufacturing, packaging, shipping, and/or storage of the composition. Typically, the amount of active agent will depend upon the specific active agent or agents employed. Typically, the active agent can be present up to about 80 wt. % of the composition, up to about 50 wt. % of the composition, up to about 25 wt. % of the composition, or up to about 10 wt. % of the composition. Preferably, the active agent can be present up to about 5.0 wt. % of the composition, up to about 1.0 wt. % of the composition, or up to about 0.5 wt. % of the composition.

When the composition is in use (i.e., when the composition is placed upon the skin of a patient (e.g., human)), the active agent can be in continuous contact with the skin surface of the patient.

In an embodiment of the invention, the amount of active agent present in the composition can be up to about 5.0 wt. % of the composition, up to 4.0 wt. % of the composition, up to 3.0 wt. % of the composition, up to 2.0 wt. % of the composition, up to 1.0 wt. % of the composition, or up to about 0.5 wt. % of the. composition. Preferably, the active agent and amount thereof will comply with FDA regulations (e.g., 2 1 C.F.R. Chapter 1, Section 333, Subpart D-Topical Acne Drug Products, Apr. 1, 2000 Edition).

Other suitable active agents are disclosed, e.g., in Physician's Desk. Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; and references cited therein.

It is within the skill of a person of ordinary skill, based on the disclosures herein, to select a ratio of solvent to water and a concentration of drug or active agent such that the drug will be present in the inventive formulation both in dissolved form and in microparticulate solid form.

The ratio of solid active agent to dissolved active agent can be six or less. A composition having a solid to dissolved active agent ratio of less than two may provide the greatest amount of pharmaceutical available for immediate partition out of the stratum corneum and into the viable epidermis. This should provide minimum reservoir capacity, but may not maintain sustained delivery or provide maximum activity in the supracorneum zone. A composition having a solid agent to dissolved agent ratio of two or greater may have a reduced amount of active agent available for immediate partition out of the stratum corneum and into the viable epidermis. This provides maximum reservoir capacity, and maintains sustained delivery, providing maximum activity in the supracorneum zone. For the present invention, the ratio for solid drug to dissolved drug should be no greater than 50, or no greater than 10, and/or no greater than six. Drug delivery from the solid/dissolved pharmaceutical formulation may be optimized to provide higher levels of active agent to the supracorneum zone, while maintaining the level of active agent partitioning out of the stratum corneum and into the viable epidermis, despite 10-fold increases in the amount of pharmaceutical applied to the skin.

In various embodiments, the topical SMP formulation is a semi-solid aqueous gel. In various embodiments, the SMP formulation includes a thickener at a concentration effective to cause formation of a gel. The formulations of the invention can be prepared using a variety of emulsifying and thickening agents well-known to those skilled in the art, such as carbomer polymers and cellulosic polymers. Polymer thickeners that may be used include those known to one skilled in the art, such as hydrophilic and hydroalcoholic gelling agents frequently used in the cosmetic and pharmaceutical industries. Preferably, the polymer thickener comprises “CARBOPOL®” (B. F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE” (ISP Technologies, Wayne, N.J.). Preferably, the thickener/gelling agent comprises between about 0.2% to about 4% by weight of the composition. More particularly, the preferred compositional weight percent range for “CARBOPOL®” is between about 0.5% to about 2%, while the preferred weight percent range for “NATROSOL®” and “KLUCEL®” is between about 0.5% to about 4%. The preferred compositional weight percent range for both “HYPAN®” and “STABILEZE” is between about 0.5% to about 4%.

The formulations can include a polymer in about 0.2 wt. % to about 10.0 wt. % of the composition. More specifically the formulation can include a polymer in about 0.5 wt. % to about 5.0 wt. %. More specifically, the formulation can include a polymer in about 0.75 wt % to about 2.0 wt %. The formulation can include a copolymer in about 0.85 wt. % of the composition.

“CARBOPOL®” is one of numerous cross-linked acrylic acid polymers that are given the generally adopted name of carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other gelling polymers that can be used in a method of the invention include hydroxyethylcellulose, hydroxypropylcellulose, cellulose gum, MVA/MA copolymers, MVE/MA, decadiene crosspolymer, PVM/MA copolymer, or a combination thereof.

The methods of the invention can include the addition of one or more cross-linked copolymers of acrylic acid at a concentration sufficient to form a gel. In the formulation, the copolymer can act as an emulsifying agent and as viscosity-increasing agent. Cross-linked copolymers for use in the present invention include those cross-polymers classified under the CTFA Cosmetic Ingredient Handbook (10th Edition, 2004) name of Acrylate/C₁₀-C₃₀ Alkyl Acrylate Crosspolymer, which is defined as a copolymer of C₁₀-C₃₀ alkyl acrylates and one or more monomers of acrylic acid, methacrylic acid or one of their simple esters crosslinked with an allyl ether of sucrose or an allyl of pentaerythritol. Cross-linked copolymers for use in the present invention also include those polymers classified under the NF monograph [USP28/NF23, 2005] for Carbomer Copolymer which is defined as a high molecular weight copolymer of acrylic acid and a long chain alkyl methacrylate cross-linked with polyalkenyl ethers of polyalcohol. Cross-linked copolymers for use in the present invention also include those polymers classified under the NF monograph for Carbomer 1342 which is defined as a high molecular weight copolymer of acrylic acid and a long chain alkyl methacrylate cross-linked with allyl ethers of pantaerythritol.

Specific examples of suitable copolymers for use in the present invention include Pemulen® TR-1 (Acrylates/C10-30 Alkyl Acrylate Crosspolymer-Noveon; noted to meet the USP 25/NF 20 monograph for “Carbomer Copolymer Type B”) and Pemulen® TR-2 (Acrylates/C10-30 Alkyl. Acrylate Crosspolymer-Noveon; noted to meet the USP 25/NF 20 monograph. for “Carbomer Copolymer Type A”), Carbopol® 1342 and 1382 (Acrylates/C10-30 Alkyl Acrylate Crosspolymer-Noveon), Carbopol® ETD 2020 (AcrylatesIC 10-30 Alkyl Acrylate Crosspolymer-Noveon), and Carbopol® Ultrez 20 and 21. More specifically, the copolymer can be Pemulen® TR-1 or Carbopol® ETD 2020.

The copolymer of an acrylic acid can be employed in any suitable amount, provided the amount of copolymer remains stable in the composition. Specifically, the compositions can include a copolymer in about 0.2 wt. % to. about 10.0 wt. % of the composition. More specifically the composition can include a copolymer in about 0.5 wt. % to about 5.0 wt. % of the composition. More specifically, the composition can include a copolymer in about 0.75 wt %. to about 2.0 wt % of the composition. Preferably, the composition includes a copolymer in about 0.85 wt. % of the composition.

In a preferred embodiment, the composition comprises dapsone and ethoxydiglycol, which allows for an optimized ratio of solid dapsone to dissolved dapsone. This ratio determines the amount of dapsone delivered, compared to the amount of dapsone retained in or above the stratum corneum to function in the supracorneum domain. The system of dapsone and ethoxydiglycol may include purified water combined with “CARBOPOL™” gelling polymer, methylparaben, propylparaben, titanium dioxide, BHA, and a caustic material to neutralize the “CARBOPOL™”

The present formulations can include an alkali, also known as a base agent. The amount of alkali can be adjusted to change pH values of the topical compositions. The pH adjustment of the compositions of the present invention can be carried out by means of inorganic bases such as sodium hydroxide and potassium hydroxide; and organic bases such as triethylamine, diisopropanolamine, and triethanolamine (trolamine). The compositions may. have a pH of about 7, e.g. 7.2, or below about 7. In other embodiments, the. compositions of the present invention can be adjusted to have a pH below about 6.0, more specifically below about 5.5, even more specifically between about 4.0 to about 5.5, even more specifically between about 4.2 to about 5.4, or 4.4 to about 5.2, or about 4.8±0.5.

Preservatives, antioxidants, fragrances, colorants, sunscreens, thickeners, suspending agents, enhancers, and other additives required to achieve. pharmaceutically or cosmetically acceptable or preferred product may also be included. Topical solutions or suspensions are not limited to these components, since one skilled in the art will be aware of additional components useful in the formulation of topical solutions or suspensions.

The formulations of the present invention may include a preservative. A preservative is useful for preventing bacterial growth, mold growth, fermentation, and/or decomposition. As used herein, “preservative” refers to any substance which prevents bacterial growth, mold growth, fermentation, and/or decomposition. Concise Chemical and Technical Dictionary, 4th enlarged. edition, Chemical Publishing Co., Inc., NY, N.Y. p. 939 (1986). Any suitable preservative can be employed, provided the preservative effectively prevents bacterial growth, mold growth, fermentation, and/or decomposition; and the preservative remains stable in the composition. Preferably, the stability is over a prolonged period of time, e.g., up to about 2 years, up to about 1 year, or up to about 6 months, typically experienced in the manufacturing, packaging, shipping, and/or storage of the composition.

Suitable preservatives include, e.g., quat-15, parabens including methyl paraben, propyl paraben and butyl paraben, chloroxylenol, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, DMDM Hydantoin, 3-Iodo-2-Propylbutyl carbamate, chlorhexidine digluconate, chloromethyl methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, and medicinally acceptable salts thereof. Preferably, the preservative is quat-15, which is commercially available from Dow Chemica. (Midland Michigan); methyl paraben; propyl paraben; ascorbic acid; or a combination thereof. In a preferred embodiment, the preservative is methyl paraben.

The preservative can be employed in any suitable amount provided the amount of preservative effectively prevents bacterial growth, mold growth, fermentation, and/or decomposition and the effective amount of preservative remains stable in the composition. In one embodiment, the preservative can be present up to about 20.0 wt. % of the composition, up to 5.0 wt. % of the composition, or up to 1.5 wt. % of the composition. The amount of preservative present in the composition will typically depend upon the specific compound or compounds employed as the preservative. For example, methyl paraben can be employed in about 0.01 wt. % to about 1.5 wt. % of the composition, in about 0.05 wt. % to about 0.50 wt. % of the composition, or in about 0.06 wt. % to about 0.25 wt. % of the composition. In a preferred embodiment, methyl paraben is employed in about 0.2 wt. % of the composition.

Emulsifiers that may be added to the composition include, but are not limited to, steareth 20, ceteth 20, sorbitan sesquioleate, sorbitan mono-oleate, propylene glycol stearate, dosium lauroyl sarcosinate, polysorbate 60, or combinations. Antioxidants, fragrances, colorants, sunscreens, thickeners, and other additives required to achieve pharmaceutical or cosmetically acceptable or preferred product may also be included. However, topical creams and lotions are not limited to these components since one skilled in the art will be aware of additional components useful in the formulation of topical creams and lotions.

The compositions of the present invention may further comprise other optional ingredients that may modify the physical, chemical, cosmetic or aesthetic characteristics of the compositions. The compositions may also further comprise optional inert ingredients. Many such optional ingredients are known for use in topical compositions, including anti-acne compositions, and may also be used in the topical aqueous compositions herein, provided that such optional materials are compatible with the essential materials described herein, or do not otherwise unduly impair product performance.

In one embodiment, the SMP formulation comprises about 0.5% to 4.0% Carbomer. In an embodiment, the SMP formulation comprises about 0.85% carbomer; about 66.95% water; about 25% diethylene glycol monoethyl ether (i.e., ethoxydiglycol); about 0.2% methylparaben; about 5% dapsone; and about 0.2% sodium hydroxide.

In another embodiment, the SMP formulation comprises about 1% carbomer, about 80-90% water, about 10% ethoxydiglycol, about 0.2% methylparaben, about 0.3% to 5.0% dapsone including both solid dapsone and dissolved dapsone, and about 2% basic agent. More particularly, the carbomer may include “CARBOPOL™ 980.”

The above-described formulations can all be prepared by an embodiment of a method of the invention. Various embodiments of the method comprise a method of preparing a solvent-microparticle (SMP) topical gel formulation comprising a bioactive drug, wherein the formulation comprises the drug dissolved in a liquid and the drug in a microparticulate solid form dispersed in the liquid, the method comprising; first, forming the liquid by combining an organic solvent and water; then, contacting the drug in a microparticulate solid form with the liquid, such that the microparticulate solid form does not entirely dissolve in the liquid; and dissolving a thickener in the liquid at a concentration sufficient to form a gel.

In various embodiments, the method further comprises, prior to the step of contacting the microparticulate solid form with the liquid, forming a solution of the drug in the liquid, wherein the drug is substantially completely dissolved in the liquid.

The water and the solvent can be combined by any of the mixing techniques well known in the art, such by stirring the water and the solvent together in a reactor of suitable size, optionally with heating, preferably gentle heating to a temperature no greater than about 50° C., then subsequent cooling to room temperature. The water and the solvent should maintain a single phase at about room temperature. It is within ordinary skill for a practitioner of the art to select the solvent and the relative proportions of the solvent and water in forming the liquid to provide a single-phase liquid. Optionally, a drug can be mixed with the liquid in a concentration such that the drug is substantially completely dissolved, or the liquid can be filtered, centrifuged, or the like to remove any undissolved drug. At this stage in the method, the liquid is homogenous with no undissolved solids. As discussed above, any of a number of different solvents can be employed, or mixtures thereof, in forming the liquid.

After the liquid comprising the solvent or solvents and water is prepared, optionally containing a drug in dissolved form, the liquid is then brought into contact with a drug in solid, microparticulate form. The drug in solid, microparticulate form can have previously been milled or ground to provide a solid form wherein the individual solid particles are of micron size, that is, are of an average particle diameter of less than 1 mm, or of less than 100 microns, or of less than 10 microns, and the sample of the solid includes substantially no particles of greater than 1 mm individual diameter. The identity and the quantity of the drug in solid microparticulate form is selected, and the quantity and composition of liquid is selected, such that the drug in solid microparticulate form does not entirely dissolve in the liquid, but rather, the drug is present in both dissolved and in solid dispersed physical form, as described above. Once the drug in solid microparticulate form is contacted with the liquid, substantially o changes are made in the composition, such as addition of large. amounts of additional water, that would bring about precipitation of the drug that is present in the dissolved physical form.

A thickener is added to the liquid at achieve a final concentration sufficient to bring about formation of a gel. The thickener can be added before or after the step of contacting the drug in solid microparticulate form with the liquid. The thickener can be a polymer as described above.

In an embodiment, a method for preparing a topical SMP formulation having dissolved and microparticulate dapsone comprises the following steps: a polymer thickener component is prepared by charging 66.95 grams of purified water to a vessel suitable to contain 100 grams of finished semisolid product, and slowly sifting 0.85 g of “CARBOPOL® 980” into a vortex formed by. rapidly stirring the purified water. When a homogeneous dispersion of “CARBOPOL® 980” and water is formed, stirring is reduced to minimize air entrapment. Next, 25 g of ethoxydiglycol, and 0.2 g of methylparaben are added to the water solution. Then, 5.0 g dapsone, which can be dapsone of polymorph Form III, in microparticulate solid form is exposed to the thickened liquid. Then, 2.0 grams of a 10% w/w aqueous sodium hydroxide solution are added to neutralize the CARBOPOL® 980 and form the gel. An excess of the solid active agent is present in the solvent system, such that excess solid remains after the solvent system is saturated.

The relative percentages for each of the reagents used in the present invention may vary depending upon the desired strength of the target formulation, gel viscosity, and the desired ratio of microparticulate to dissolved drug. It is within ordinary skill, using the disclosure herein, to select reagents and their relative proportions to prepare a topical SMP formulation by a method of the invention. Unless otherwise indicated, all reagents listed above are commonly known by one of ordinary skill in the art and are commercially. available from pharmaceutical or cosmetic excipient suppliers.

The formulations prepared by the method described herein are useful for treating conditions of the skin. In an embodiment, the formulations are used to treat acne. In other embodiments, formulations prepared by methods of the present invention are used to treat dermatological conditions such as impetigo, erythrasma, erysipelas, rosacea (perioral dermatitis, rhinophyma), furuncles, carbuncles, alopecia, panniculitis, psoriasis, dermatitis, cysts, bullous diseases (pemphigus vulgaris, bullous pemphigoid, and herpes gestationis), collagen vascular diseases (dermatomyositis, systemic lupus erythematosus, eosinophilic. fasciitis, relapsing polychondritis, and vasculitis), sarcoidosis, Sweet's disease, lichen planus, hirsutism, toxic epidermal necrolysis, dermatitis herpetiformis, eczema, atopic dermatitis, seborrhoeic dermatitis (dandruff, cradle cap), diaper rash, urushiol-induced contact dermatitis, erythroderma, lichen simplex chronicus, prurigo nodularis, itch, pruritus ani, nummular dermatitis, dyshidrosis, pityriasis alba, parapsoriasis (pityriasis lichenoides et varioliformis acuta, pityriasis lichenoides chronica), pityriasis rosea, pityriasis rubra pilaris, urticaria (dermatographic urticaria, cholinergic urticaria), erythema (erythema nodosum, erythema multiforme, Stevens-Johnson syndrome, toxic epidermal necrolysis, erythema annulare centrifugum, erythema marginatum), sunburn, actinic keratosis, polymorphous light eruption, radiodermatitis, erythema ab igne, nail disease, onychogryposis, Beau's lines, yellow nail syndrome, follicular disorders, alopecia areata (alopecia universalis), androgenic alopecia, telogen effluvium, lichen planopilaris, trichorrhexis nodosa, hypertrichosis (hirsutism), epidermoid cysts, sebaceous cysts, pseudofolliculitis barbae, hidradenitis suppurativa, miliaria, anhidrosis, body odor, chromhidrosis, vitiligo, melasma, freckles, caféau lait spots, lentigo/liver spots, seborrheic keratosis, acanthosis nigricans, callus, pyoderma gangrenosum, bedsores, keloids, granuloma annulare, necrobiosis lipoidica, granuloma faciale, morphea, calcinosis cutis, sclerodactyly, ainhum and livedoid vasculitis.

All of the publications cited hereinabove are incorporated by reference herein. The invention has been described with reference to various specific embodiments and techniques. However, it should be understood that many variations and modifications may be made while remaining within the spirit and scope of the invention.

What is claimed is:

1. A method of preparing a solvent-microparticle (SMP) topical gel formulation comprising a bioactive drug, wherein the formulation comprises the drug dissolved in a liquid and the drug in a microparticulate solid form dispersed in the liquid, the method comprising:
first, forming the liquid by combining an organic solvent and water; then;
contacting the drug in a microparticulate solid form with the liquid, such that the microparticulate solid form does not entirely dissolve in the liquid; and
dissolving a thickener in the liquid at a concentration sufficient to form a gel.

2. The method of claim 1 further comprising, prior to the step of contacting the microparticulate solid form with the liquid, forming a solution of the drug in the liquid, wherein the drug is substantially dissolved in the liquid.

3. The method of claim 1 wherein the amount of the drug in microparticulate solid form dispersed in a unit volume of the liquid is no more than about six times the amount of the drug dissolved in the unit volume of the liquid.

4. The method of any one of claim 1 wherein the topical gel formulation comprises a preservative, an active surfactant, an emulsifier, an antioxidant, or a sunscreen, or any combination thereof.

5. The method of claim 1 comprising, after the step of forming the liquid, adding a preservative, an active surfactant, an emulsifier, an antioxidant, or a sunscreen, or any combination thereof, to the liquid.

6. The method of claim 3 wherein the drug is dapsone.

7. The method of claim 1 wherein the solvent comprises diethyleneglycol monoethyl ether (DGME), N-methylpyrrolidone (NMP), N,Ndimethylformamide. (DMF), N,N-dimethylacetamide (DMA), or dimethylsulfoxide (DMSO), or any combination thereof.

8. The method of claim 1 wherein the thickener comprises a carbomer.

9. The method of claim 8 wherein the carbomer is Carbomer 980.

10. The method of claim 6 wherein the drug is present in the formulation at a total concentration of about 3-5%.

11. The method of claim 6 wherein the topical formulation comprises a preservative, the preservative being dissolved or dispersed in the gel.

12. The method of claim 11 wherein the preservative comprises methyl paraben.

13. The method of claim 11 wherein the topical formulation further comprises an alkali.

14. The method of claim 13 wherein the alkali is sodium hydroxide or potassium hydroxide.

15. The method of claim 11 wherein the topical formulation has a pH of about 7.0-7.6.

16. The method of claim 6 wherein the drug in a solid form in the gel comprises dapsone Form III.

17. The method of claim 6 wherein a second drug is present in the formulation.

18. The method of claim 17 wherein the second drug is present in a dissolved form.

19. The method of claim 18 wherein the second drug is present in a microparticulate solid form.

20. The method of claims 19 wherein the second drug comprises a glucocorticoid, an antibiotic agent, an antiseptic, an acidic compound, or a retinoid, or a combination thereof.
